(19) United States
(12) Patent Application Publication
(10) Pub. No.: US 2004/0068421 A1
(43) Pub. Date: Apr. 8, 2004

(54) Patient station with integrated customer support

(76) Inventors: Georges Drapeau, Quebec City (CA); Martin-Pierre Roy, Quebec City (CA); Serge Beaudry, Quebec City (CA); Jean-Luc Morin, Quebec City (CA); Kevin Bouchard, Quebec City (CA); Hugo St-Pierre, Quebec City (CA); Daniel Huot, Quebec City (CA); Nathaniel Findlay, Quebec City (CA); Katharine Ouellet, Quebec City (CA)

Correspondence Address: FOLEY AND LARDNER, SUITE 500, 3000 K STREET NW, WASHINGTON, DC, 20007, US

(21) Appl. No.: 10/417,418

(22) Filed: Apr. 16, 2003

Related U.S. Application Data
(60) Provisional application No. 60/372,678, filed on Apr. 16, 2002.

(51) Int. Cl. G06F 17/60; G06F 15/16

(52) U.S. Cl. 705/2; 709/203

(57) ABSTRACT

A communications apparatus has an interface unit, interface unit with at least one input/output device and a computer. The input/output device is responsive to the computer to present clinical information to a care giver and non-clinical information to a patient. At least one server operatively connected to the interface unit provides customer services to assist the care giver and patient to use the apparatus. The system automatically guides the user through the process required to accomplish the desired result. Where the user prefers, as indicated by the user's activation of a “operator assist” feature or where the system cannot resolve the user's inquiry in the absence of operator assistance, the user is connected to a live operator. Such a connection is made using voice over internet or other techniques, so that it is not necessary to provide a separate telephone connection. The operator can take control of the user's computer and can communicate both visually and verbally with the user as required.

### CROSS-REFERENCE TO RELATED PATENT APPLICATIONS

This application claims priority to U.S. Provisional application 60/372,678 filed on Apr. 16, 2002, the contents of which are incorporated herein by reference.

### FIELD OF THE INVENTION

The invention relates to communications systems and entertainment systems in general. In particular, the invention relates to an apparatus and method for providing a bedside station that allows a patient to access media and services, while at the same time allowing care givers to access clinical information concerning with the patient or the patient's condition. More specifically, the invention provides integrated customer service that, when accessed by the user in such an environment, provides an appropriate level of response to user inquiries, including connecting the user to a manned operations center.

### BACKGROUND OF THE INVENTION

U.S. Pat. No. 5,867,821 to Ballantyne et al discloses an automated system for the distribution and administration of medical services, entertainment services, electronic health records and the like for hospitals, other health care facilities, including the patient's bedside in a hospital or at the patient's domestic premises. Ballantyne discloses a master library, a communications interconnection system associated with the master library, an automated nursing station, and an electronic patient care station with a monitor, an interface to communicate with the master library and the nursing station, an input entry device and compression and decompression means for data passed to the patient station. Ballantyne does not provide a facility for assisting users in the use of such systems.

### SUMMARY OF THE INVENTION

According to the invention a communications apparatus has an interface unit with at least one input/output device and a processor or computer. The input/output device is responsive to the processor or computer to present clinical information to a care giver and non-clinical information to a patient. For example, a patient can use a device according to the invention to access the internet, to order meals, to access entertainment or to conduct video conferencing. A server operatively connected to the interface unit provides customer services to assist the care giver and the patient to use the apparatus. In one aspect of the invention, a care giver using such a bedside patient station apparatus according to the invention may access the server through a customer service interface in order to obtain assistance in accessing patient medical records, to obtain clinical information about diagnosis consistent with symptoms the patient presents, or to access care giver standards for a particular situation. A patient may access the server for assistance in obtaining video on demand or other services. In each case; according to the invention, the system automatically guides the user through the process to accomplish the desired result. Where the user prefers, as indicated by the user's activation of an “operator assist” feature, or where the system cannot resolve the user's inquiry in the absence of operator assistance, the user is connected to a live operator. Such a connection is made using, for example, voice over internet techniques, so that it is not necessary to provide a separate telephone connection. The operator can take control of the user's computer and can communicate both visually and verbally with the user as required.

### BRIEF DESCRIPTION OF THE DRAWINGS

FIG. 1 is a diagram showing a global view of a hardware configuration according to the invention.

FIG. 2 is a more detailed illustration of a generic patient station network according to the invention.

FIG. 3 provides a logical view of a patient station network according to the invention.

FIG. 4 illustrates an implementation of a single server operational scenario according to the invention.

FIG. 5 illustrates an implementation of a two server operational scenario according to the invention.

### DETAILED DESCRIPTION OF THE PREFERRED EMBODIMENTS

A system according to the invention includes a communications station that can be located in a facility, such as a health care facility, to provide a wide variety of media access options. For example, such a station could be located in public waiting areas of the facility to allow those waiting for service to view media content available as scheduled or on demand or to access other services through a communications network, such as the Internet. Use of the station could be provided as a courtesy or for a fee, for example based on usage and content viewed.

Another use for such a system is at a patient's bedside. A communications system according to the present invention forms a patient station that allows a patient to interact with a care giving facility, such as a hospital, and the care giving facility to interact with the patient. A patient station according to the invention also allows the patient to interact with services outside the care giving facility. According to an integrated customer service aspect of the invention, a user seeking assistance with operating the system can be connected to a manned customer operations center for personalized assistance without the need to make a separate telephone call.

An apparatus according to the invention has an input/output interface unit, such as a separate keyboard and display or a single touch sensitive screen used to detect and process user inputs and display outputs to the user, at each bedside. Such a device allows a patient to access entertainment, information and services and allows a care giver to access clinical and other information. The apparatus according to the invention may incorporate or be used with bar code scanners, audio speakers, microphones, cameras, video conferencing equipment, game consoles and other auxiliary devices without departing from the invention. Clinical information accessed through the patient station, typically by a caregiver, could include patient specific information, such as patient test results, as well as published information, such as drug interaction data, recommended treatment regimes and care facility standards and procedures. A bedside information gateway to relevant databases assists the care giver in accessing such information. The care giving facility can use the system to manage and monitor patient treatment and medical condition parameters, to maintain patient histories and charts, to avoid dispensing incorrect drug prescriptions or dosages, to highlight and require approval of deviations from procedures and treatments that are considered standard in the science of medicine or are mandated by the facility's practice standards, to assist in diagnosis and to provide the patient with information tailored to the patient's condition. An apparatus according to the invention could also restrict access to such clinical information based on authorization criteria.

According to the invention, when a patient communications station is not in use to display clinical information to a caregiver at the bedside, the system incorporates features to allow the patient to use the patient station to access a wide range of services and media of particular use and interest to the patient. The features accessed by the patient through the system could be services provided by the care giving facility or by another party. Such other parties could include a contracted service that provides access to media on demand or other entertainment services or Internet access. In one example, the patient uses the system to review media files that reside on the care giving facility's server and are relevant to the patient's condition. Such files could include information that the patient needs to understand and cope with his or her medical condition. In other examples, the patient accesses other services offered by the care giving facility, such as ordering meals. In this case, where the patient's condition restricts the patient's dietary options, the system can be programmed to offer the patient only those options suitable to the patient's condition or to reject inappropriate selections. In either case, the system according to the invention could suggest alternative selections to the patient. These features could be implemented in a particularized manner by storing and accessing a dietary profile for each patient or, in a more general manner, by retrieving the patient's current condition and comparing that condition with information stored in a database of dietary recommendations and restrictions related to different conditions. The database could be a proprietary database stored at the care giving facility or could be a database the care giving facility accesses through a network.

According to the invention, the patient can also use the system to access features available from sources outside the care giving facility. For example, the system is connected so that the patient can access the Internet through a contracted Internet service provider. This permits the patient full access to his or her personal e-mail and allows the user to connect to an employer's network. The system according to the invention also incorporates equipment that allows the patient access to televised programming and video on demand services.

Another feature according to the invention is access to an Integrated Customer Support (ICS) feature. According to the invention, a user having questions about an aspect of the system accesses the ICS feature. Software operates to instruct the user through a series of HELP screens, until the system resolves the user's inquiry. Where the system cannot resolve the inquiry or where the user chooses to activate a personalized assistance feature, the system according to the invention allows the user to connect to a manned customer service center, using for example, voice over Internet Protocol technology. Thus, according to the present invention, a user inquiry can begin with a help inquiry that activates programmed software to respond automatically to conditions it observes, query the user for information and instruct the user about proper steps to accomplish the desired result. When appropriate, the system can automatically connect the user to a manned customer support center that assists the user in resolving user inquiries. In order to accommodate privacy concerns, the system can also be programmed to connect the user to a manned station only after receiving confirmation from the user during the problem resolving session. Alternatively, the system according to the invention can be programmed to allow the user to activate at any time an “assist” feature that connects the user to a manned operations center, such as a network operations center, to provide customer assistance. In this way the human response from a manned customer support center becomes integrated with the apparatus itself.

In another aspect of the invention, the system monitors its own performance and alerts the network operations center when the system detects a degradation or requires repair. According to the invention, the network operations center remotely monitors and manages the performance of the system's hardware and software. This remote monitoring and repair avoids unexpected downtime and frees the care giving facility from the responsibility of attending to the system.

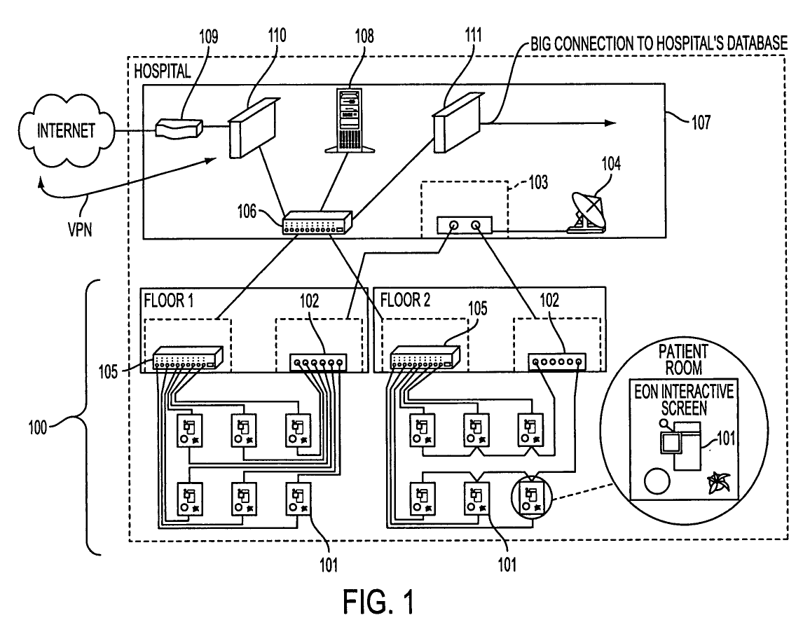

FIG. 1 is a simplified illustration of a system according to the invention. FIG. 1 shows a system architecture 100 having three layers. First, at a patient layer 100, patient station 101 has an interactive screen or other interactive input/output devices, and a computer, such as a thin client computer, located in a patient's room. The patient station 101 may also include mounting devices (not shown) to install the patient station 101 near the patient's bedside within the patient's reach and connections to a network of processors and servers (via a data closet or local area network (LAN) switch 105) which provide information and services. For illustration purposes and not by way of limitation, FIG. 1 shows six patient stations 101 on each of two floors. The patient stations 101 are connected to a video splitter/amplifier 102 which provides a connection to a television head-end video splitter/amplifier 103 receiving television programming, for example, from a satellite or cable service 104. This separate connection to such services allows a care giving facility to freely contract such services from those available on competitive business terms.

A group of patient stations 101, for example, the patient stations 101 on one floor or part of one floor, connect to the LAN switch 105. As shown in FIG. 1, the various LAN switches 105 connect to another LAN switch 106 which provides access to the next layer of the hierarchy, the data center layer 107, which typically provides data and communications management at the care giving facility. Data center layer 107 has a local media server 108 which may contain specialized media for the facility. For example, media server 108 could provide patients access to descriptions of hospital services or could contain care management information that the care giving facility prescribes for patients with certain conditions. The data center layer 107 also contains a router 109 accessed through a firewall 110 to provide users access to external computer networks, such as through an Internet Service Provider. Using conventional techniques, users can then access their own preferred internet services. Switch 106 also provides access through firewall 111 to a third level of the hierarchy, the hospital's database (not shown). Such access is provided through a Bedside Information Gateway (BIG) and allows care givers and administrators to access patient data, hospital treatment standards, medical information, test results and other important information.

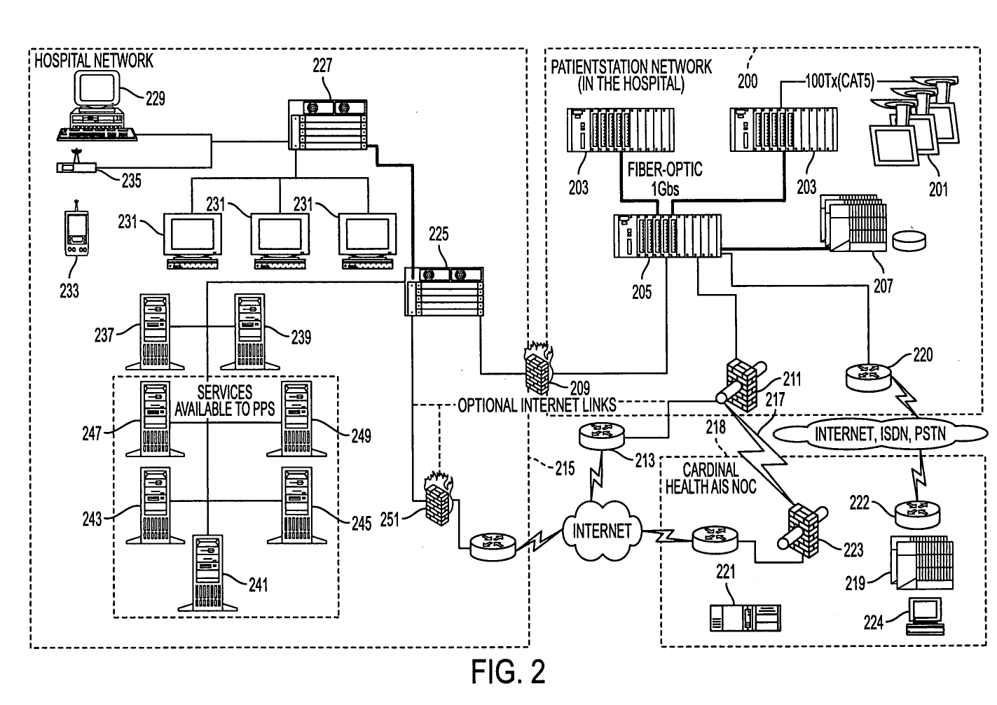

FIG. 2 is a more detailed view of a patient station network 200, which may be in a hospital, according to the invention. In FIG. 2, patient stations 201 are connected to switches 203, such as commercially available Procurve 2524 switches or 100 BaseT switches. Switches 203 are routed to backbone switch 205, such as a commercially available HP 4108 GL switch. Servers 207 provide content, Voice over Internet Protocol (VoIP) telecommunication (for example to communicate with a customer support representative, as discussed further herein), billing and radio. The backbone switch 205 is connected through BIG Firewall 209 and Internet Firewall 211 to provide bedside internet access via internet service provider router 213 and access to the care giver or hospital network 215. Access to a Virtual Private Network (VPN) 217 is also provided through Internet Firewall 211. The Virtual Private Network 217 provides access to a Network Operations Center (NOC) or Data Center 218 that includes servers 219 and monitoring station 221 to assist in providing operational functions of the patient station network 200 itself. Such functions include monitoring the status of the network 200 and identifying potential maintenance issues. Another possible function is to provide access to customer assistance features. A voice gateway 220 in the patient station network 200 interfaces with a voice gateway 222 in network operations center 218 to provide a voice interface between the user of the patient station 201 and a customer support representative or agent. Computer 224 in the network operations center 218 is used by the customer support representative to communicate through the voice gateways 220, 222 with the user of patient station 201 through various media including the Internet, Integrated Services Digital Network (ISDN) lines or the telephone network (PSTN). The network operations center 218 can also access the Internet or other computer network through Firewall 223.

Care giver or hospital network 215 may be a network existing prior to the installation of a patient station network 200 with customer assistance according to the invention. By way of example, and not limitation, care giver network 215 is shown in FIG. 2 as a hospital network, although other care giver networks 215 could be used. Hospital network 215 includes hospital switches 225 and 227 which provide access to the hospital network 215 through hospital computer terminals 229, specialized stations 231, mobile devices 233, other access points 235 or through the patient station 201 via the Bedside Information Gateway. The hospital network 215 would typically include clinical servers 237 and an Admission and Discharge Transfer (ADT) system 239. A variety of other servers could also be accessed by the patient station 201 through the network 215, such as a Web server 241 hosting patient education information and other world wide web (WWW) applications, a clinical applications server 243 accessed by authorized hospital personnel, mainframe and mini unix servers 245, a server 247 for Windows applications, and an admission and discharge transfer (ADT) interface server 249, accessed by applications as necessary. The hospital could have its own internet access through hospital firewall 251, which optionally could be accessed through the patient stations 201, as well.

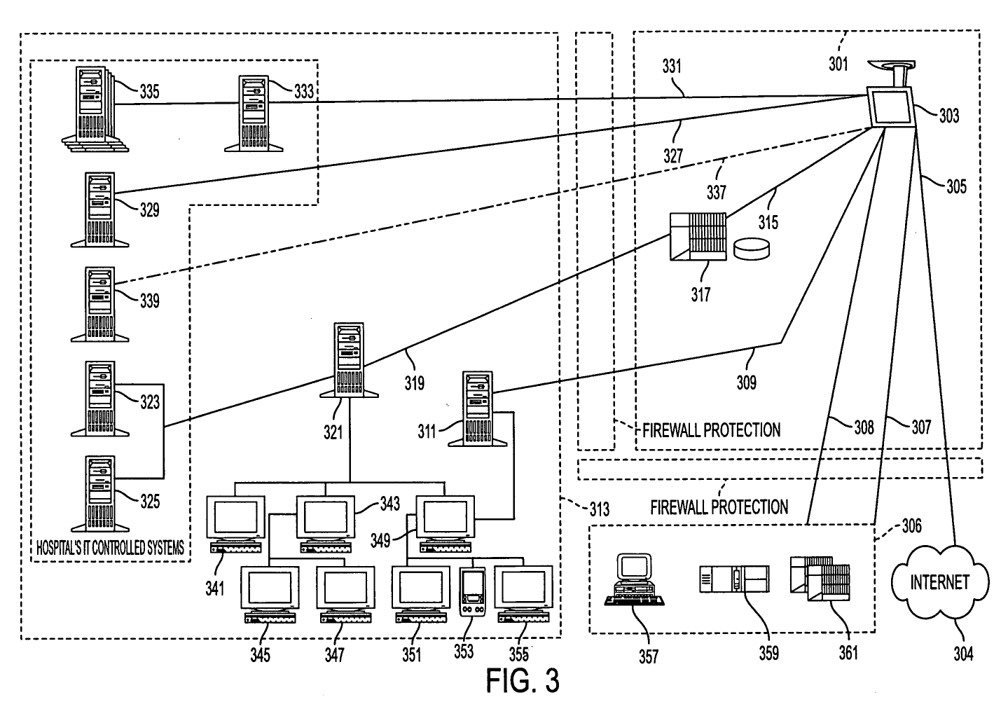

FIG. 3 is a logical diagram showing information traffic in a such a system. As illustrated in FIG. 3, the patient station network 200 of FIG. 2 can be implemented separately and distinctly from an existing hospital network 215 to provide an informational interface that takes advantage of capabilities already in place at the care giver facility. Patient station network 301 has several informational transfer interfaces. A patient station 303 can communicate using HTTP protocols to the Internet 304 on informational interface 305 (communication may be content filtered) or to the network operations center 306 on informational interface 307 (monitoring, update, remote control, logs, billing traffic (VPN), or Voice over Internet Protocol informational interface 308. NOC 306 may be similar to NOC 218 in FIG. 2 and can include a customer support representative computer 357, a monitoring station 359 and serves 361. The remaining informational interfaces are to the care giver or hospital network. For example, using informational interface 309, authorized care giver personnel can use the patient station 303 to communicate (thin client or web traffic) with a clinical applications server 311. Within the care giver network, the clinical applications server 311 can communicate with a variety of consoles and devices, as shown generally at 313 connected to server 311. These consoles and devices include, for example, a packager computer 341, a supply console 343, supply stations 345 and 347, a medical console 349, a medical station 351, a PDA 353 and an anesthesia system 355. The patient station 303 also communicates over information interface 315 to patient station network servers 317 that provide services such as video on demand (VoD), VolP, radio and billing within the patient station network 301. Servers 317 may also communicate with the care giver network over communications interface 319, for example, through a linking device 321 that links the patient station 303 to a variety of care giver facilities. These facilities could include a pharmacy information system 323 to which pharmacy data is provided and a hospital ADT system 325 to which ADT messaging is provided. The patient stations 303 also communicate using HTTP protocols over informational interface 327 to care giver web servers 329. Other thin client traffic from the patient station 303 can be transmitted over informational interface 331 through a Citrix server 333 that provides access to non-WWW clinical applications or a non-WWW clinical server 335 that provides access to facilities such as meal ordering. Finally, where appropriate, a patient station 303 can communicate terminal traffic over informational interface 337 with a mainframe or mini unix server 339, as shown in FIG. 3.

According to the invention, integrated customer services are provided through a Customer Operations Center or a Network Operations Center. The Customer Operations Center is typically manned at scheduled times, preferably 24 hours a day, in order to assist system users in accessing available services and features. In operation, the user activates a HELP feature through the patient station's user interface device. The HELP feature is context sensitive and determines the type of operation that the user is performing at the time the user accesses the HELP feature. Upon making this determination, a processor at the Customers Operations Center transmits relevant information to the inquiring patient station.

An integrated customer service station according to the invention facilitates the customer's use of a patient station because it avoids the need for the patient to use an additional peripheral such as the traditional phone to request help and because it provides the customer a real time demonstration of the solution to his problem. Indeed, a system according to the invention can have the additional advantage of completely replacing the room phone and can provide previously unavailable communications capabilities. For example, using the integrated customer service according to the invention, a customer could call another patient station and legacy phone users, receive calls and voice messaging, redirect call and messages to a mailbox and request online help from customer support. Those of ordinary skill will recognize that other features, such as the file transfer and chat features, could also be parts of Integrated Customer Service (ICS) according to the invention.

ICS according to the invention uses VolP (voice over internet protocol) technology to offer the user communications features integrated within a patient station. One example is ICS based on the H.323 VolP protocol. H.323 has been developed by the International Telecommunication Union (ITU) to transfer voice and video throughout Internet Protocol networks.

Remote access features of the invention can be implemented using VNC to provide a real time solution to a problem in response to a customer's request from the support representative. VNC is an open source server software application program used commonly to provide remote assistance to inexperienced users. Responding to dialog screens, one can upload a file from a remote computer to a server or to download a file from a server to a remote computer. Using VNC, according to the invention remote activation can also be used to modify a current session, grant access to certain media, modify time left to access media, activate or deactivate media or prompt messages to the user of the patient station. In accordance with the invention, the system is programmed to identify the type of user and media for which access is sought and to respond accordingly. For example, the system would not permit deactivation of access to media needed to display clinical information to a care giver.

ICS provides a user-friendly web interface to implement its services. A customer can talk to an agent by simply clicking on the touch screen. The customer support representative also use a web interface to provide online help.

Behind all interfaces, an H.323 server called a gatekeeper keeps a client database and translates machine names to raw Internet addresses. This gatekeeper is also used to control bandwidth on the network to ensure security and quality of service. The gatekeeper provides proxy (traffic control and routing) features. In ICS, the gatekeeper will be used in a customer support location to ensure that the patient station user database is always updated and that voice flow is routed successfully.

Other features according to the invention include protocols and software to perform call center tasks. Such protocol and software runs on top of H.323 VolP. Encryption algorithms ensure security over Internet and local area networks. Other security techniques avoid the most likely denial of service (DoS) attacks, such as those resulting from computer hacking. HTTP protocol is used to fully manage and configure the call center.

In another configuration according to the invention, the H.323 gatekeeper server routes calls directly or based on a customer support representative request. This server takes into account languages before performing call routing, so that a client speaking Spanish is directed to a support representative that speaks this language. This server also provides music and advertisements on hold. Music and advertisements are streamed in the customer's language when all customer support representatives are busy. The customer will also be able to leave a message if he becomes tired of waiting or if no agent is signed on the system while the call center is open. After a customer leaves a message, a customer support representative calls the customer back when he or she becomes available. The call centers can be set up so that calls are routed to a preferred call center during those hours that the call center is operational and re-routed to a different call center at other times. The user or customer need not be aware where the calls are being routed.

The above features are best implemented using two servers. The first server acts as an ICS main server that handles all waiting queues and dispatches calls based on agent availability. The second server is a hospital local server that communicates with the ICS main server, and provides the music and answering machine feature. This multiple server architecture provides a number of advantages. First, it allows multiple hospital support. Second, the local server provides music on hold and advertisements without the bandwidth constraints associated with a single central server. Third, when multiple servers are in use, call center clients are not totally dependant upon only one main server. For example, if the main server or local server crashes, an alternate will be ready to replace it without affecting the entire network.

Finally, multiple servers allow more than one customer support site and permit dynamically routing calls to these different sites. For example, alternative customer support sites can be used at different times. Assume that a first call center is open from 8 AM to 5 PM Monday to Friday and the second center is open 24 hours a day. The multiple server architecture allows calls to be routed to the first server during its open hours and to the second server at other times. Alternatively, customer support sites can be used in a load sharing arrangement where such re-routing could occur when all representatives at one center are busy and there is capacity to accommodate an incoming inquiry at another call center.

The two types of servers provide both same and different functionalities. This can be accomplished with a configurable application that allows one to set a processor up as a server-client or a full server. The two servers provide the centralized answering machine and secured users registration features. These secured registered users are called the “members”. Members are assigned properties such as an H.323 alias (The H.323 specification requires a global unique ID such as traditional phone networks that require phone numbers), a full name, a password, an e-mail address and an answering machine account through which the ICS client can upload a user proprietary audio welcome file. The answering machine records the caller message, encodes it in MP3 format and attaches it to an e-mail that is sent to the member's e-mail address. All passwords are encrypted and must be provided when a welcome message is uploaded and a registration or un-registration occurs.

According to the invention, the main server controls all customer support representative or agent properties, waiting queues, call recording and local voice mailboxes. Customer support representatives or agents are assigned the same properties as members, in addition to a voluntary status and a call status. The main server requires the customer support representative or agent to register with his alias and password. In an automatic mode, a call is routed when it arrives based on agent status. An agent decides the voluntary status after providing his password. If an agent sets his voluntary status to busy, no call will be redirected to that agent. In a manual mode, each call is parked in a queue until an agent decides to take it. An agent must provide his password in order to remove a call from the queue. An agent is automatically a member of the call center and also owns a voice mailbox in addition to a customer support mailbox.

Servers are secured by being programmed with information identifying those sources from which the server is permitted to accept data. Unknown IP addresses are ignored.

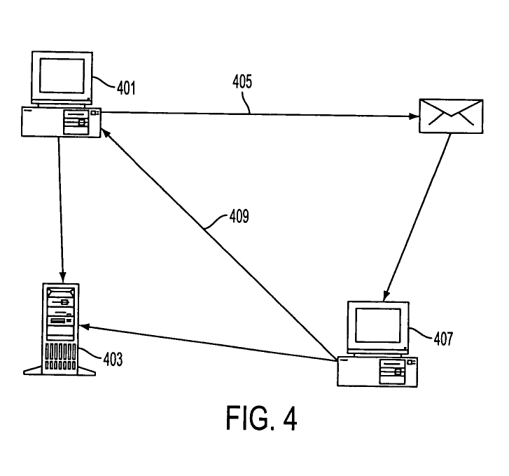

By way of illustration and not limitation, FIG. 4 illustrates one possible operational scenario according to the invention with one server in which a request for help issues from a user at patient station 401. The H.323 gatekeeper server 403 maintains and updates a database of authorized users and customer support representatives (agents). In one scenario, the customer or client requests help, such as by pressing a button labeled, “talk to a customer support representative.” The client updates the gatekeeper database of the gatekeeper server 403 and patient station 401 transmits an e-mail message as shown at 405. A customer support representative at computer station 407 receives the e-mail, reads it, and clicks on the URL in it. The representative registers with the gatekeeper server 403 and then requests the client's address, and a call is initiated and established from the customer support representative's computer 407 to patient station 401 as shown at 409.

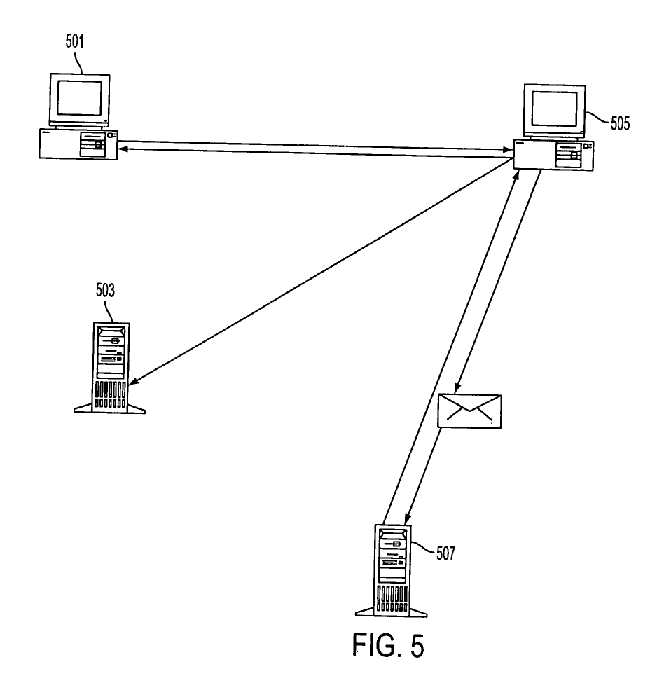

FIG. 5 illustrates an example of operation in the two server case. As shown in FIG. 5, after a patient station 501 registers itself to the H.323 gatekeeper server 503, an e-mail is sent from the patient station 501 to a customer support representative at computer station 505. The customer support representative reads the e-mail and by clicking on the link to the patient station 501 in the e-mail, causes the e-mail with corresponding request to be sent to the ICS server 507. ICS server 507 receives the request and sends to the customer support representative at computer station 505 all the data it needs to initiate communication with the inquiring patient station 501. The customer support representative at computer station 505 registers with the gatekeeper server 503 and requests the client's address. The customer support representative at computer station 505 then automatically calls patient station 501.

An other option according to the invention is to register calls for future reference. A registered call is encoded in MP3 format and classified in a subfolder with a name including the customer support representative's identifying information.

The customer support representative counsels the customer and can demonstrate to the customer how to accomplish functions desired by the customer. In order to provide this service, the customer support representative has the ability to activate and deactivate the call, to proxy into the customer's computer and take control of its operation, and to re-boot the user's computer, for example, as a step in installing additional software.

In a system according to the invention, a user views numerous screens to access different services and features of the system. As part of many such screens, a Live Support access option is presented. When a user activates Live Support, a page is displayed to notify the user that a call has been placed and the user is on hold. In actuality, an e-mail has been sent to the Customer Support Center or Network Operations Center to be picked up by a customer support representative or agent who calls the user back as discussed in the above scenarios of FIGS. 4 and 5. The user can wait and continue using the patient station while waiting or hang up. Since e-mail was sent to the Customer Support Center, a phone connection between the user and the Customer Support Representative had not yet been made. When the agent becomes available, he or she opens the e-mail and, by clicking a URL in the e-mail, causes the phone connection to be made. The user display changes to alert the user that the customer support agent is now on-line. If the user has activated another system feature, the user is returned to the call screen.

Those of ordinary skill will recognize that the invention in the appended claims may be implemented by numerous alternative computer and processing systems and that the invention is not limited by such implementation details and configurations.

What is claimed is:

1. A communications apparatus comprising an interface unit, said interface unit comprising at least one input/output device and a processor, said input/output device being responsive to said processor to present clinical information to a care giver and non-clinical information to a patient, and
a server operatively connected to said interface unit to provide customer services to assist said care giver and said patient to use said apparatus.

2. A communications apparatus as recited in claim 1, said processor being operatively connected to said input/output device and responsive thereto to control presentation of a display on said input/output device.

3. A communications apparatus as recited in claim 2, said processor comprising a client computer operatively connected to a server.

4. A communications apparatus as recited in claim 2, comprising an external video input to said processor.

5. A communications apparatus as recited in claim 2, said video input being connected to a source of programming transmitted from a transmission facility.

6. A communications apparatus as recited in claim 5, said programming comprising televised programming.

7. A communications apparatus as recited in claim 3, comprising at least one switch having a state operatively connecting said processor to said server.

8. A communications apparatus as recited in claim 7, said switch having a state operatively connecting said processor to an external computer network.

9. A communications apparatus as recited in claim 7, said switch having a state operatively connecting said processor to a database.

10. A communications apparatus as recited in claim 9, said database comprising patient records accessible to said care giver for said clinical display.

11. A communications apparatus as recited in claim 7, said switch having a state operatively connecting said processor to an integrated customer support center.

12. A communications system as recited in claim 1, said server registering said patient station and a customer support agent computer on a network.

13. A communications system as recited in claim 12, said customer support agent computer being responsive to an e-mail message from said patient station to initiate a call to said processor.

14. A communications system as recited in claim 12, comprising a second server.

15. A communications system as recited in claim 14, said customer support agent computer receiving said e-mail and passing information in said e-mail to said second server.

16. A communications system as recited in claim 15, said second server being responsive to said information to provide data necessary to allow said customer support agent computer to initiate a call to processor.

17. A communications system as recited in claim 12, said computer having stored thereon program indicia to provide said processor an ability to send messages to said input/output device station.

18. A communications station as recited in claim 17, said program indicia further comprising program indicia to provide said computer an ability to control operation of said processor.
